(19) United States
(12) Patent Application Publication
(10) Pub. No.: US 2013/0156823 A1
(43) Pub. Date: Jun. 20, 2013

(54) Excipient-free Aerosol Formulation

(71) Applicant: MAP Pharmaceuticals, Inc., Mountain View, CA (US)

(72) Inventors: Libo WU, Mountain View, CA (US); Wiwik Watanabe, Mountain View, CA (US)

(73) Assignee: MAP Pharmacauticals, Inc., Mountain View, CA (US)

(21) Appl. No.: 13/721,444

(22) Filed: Dec. 20, 2012

Related U.S. Application Data
(60) Provisional application No. 61/578,187, filed on Dec. 20, 2011.

(51) Int. Cl. A61K 9/12

(52) U.S. Cl. CPC A61K 9/12
USPC 424/400; 424/43; 514/221; 424/45

(57) ABSTRACT

Methods and compositions for producing formulations for orally inhaled benzodiazepines that do not require the presence of a surface modifier are described. The formulations are useful in the treatment of epileptic seizures.

This application claims priority under 35 U.S.C. §119(e) from U.S. Provisional Application Ser. No. 61/578,187, filed Dec. 20, 2011, which is hereby incorporated by reference in its entirety.

### TECHNICAL FIELD OF THE INVENTION

The compositions and methods described herein are in the field of orally inhaled aerosol formulations. Specifically, compositions and methods that allow for the administration of excipient-free orally inhaled formulations are described. More specifically, drug formulations that allow for oral inhalation of benzodiazepines are disclosed.

### BACKGROUND OF THE INVENTION

Aerosols are increasingly being used for delivering medication for therapeutic treatment to the lungs. This type of pulmonary drug delivery depends on the subject inhaling an aerosol through the mouth and throat so that the drug substance can reach the lungs. For drugs that are systemically active (e.g., the intended active site is not the lungs), inhalation delivery to the alveolar region of the lung is preferred.

Oral inhalation delivery of the aerosol drug formulation to the preferred region of the lungs depends on several factors. One factor is the size of the aerosol particles. Generally, for oral inhalation, the preferred particle size ranges from 0.1 microns to 10 microns. On the larger size of this spectrum, the particles tend to not reach the lungs, but instead, a large percentage of the particles get lodged in the mouth or throat, which then gets swallowed by the subject or is orally absorbed. For drugs that are intended to be systemically absorbed through the lungs, the preferred particle size of the aerosol drug formulation is in the range of about 0.5 microns to 3 microns. Particles in the smaller range of this spectrum or even smaller than 0.5 microns may be expelled before systemic absorption due to exhalation.

There are several advantages for aerosol delivery of systemically active drugs to the lungs. One major advantage is the fast absorption through the lung and delivery of the drug into systemic circulation. This advantage is particularly suitable for drugs that require a fast onset of action. Benzodiazepines are one class of molecules that fit this category. Benzodiazepines are a class of psychoactive drug that enhance the effect of the neurotransmitter gamma-aminobutyric acid (GABA). This class of drug has been used to treat anxiety, insomnia, agitation, seizure, muscle spasms, and as a sedative. Benzodiazepines such as lorazepam have been used to treat epilepsy and epileptic seizures.

Lorazepam has generally been formulated either for oral administration or for intravenous (IV) or intramuscular (IM) administration. Because of its low water solubility, both injectable formulations (i.e., IV or TM) have been difficult to formulate. Oral formulations of lorazepam have disadvantages such as susceptibility to enzyme degradation in the gastrointestinal track and oral delivery may have a slow absorption and onset of action. Oral inhalation delivery of benzodiazepines such lorazepam would overcome these difficulties and/or disadvantages.

Pressurized metered dose inhalers (pMDI) are the most common vehicles for the delivery of drugs into the lungs, accounting for approximately 65% of the total prescribed aerosols. There are two basic types of pMDI formulations: (i) solution-based, in which the active ingredients are dissolved in the propellant; and (ii) suspension-based, in which the active ingredients are suspended in the propellant. Surfactants and other surface-modifying agents are typically used in suspension formulation because suspension in the propellants is inherently unstable due to the cohesive forces between particles and due to the gravitational fields. Therefore, surfactants and other surface-modifiers are generally required in order to provide stability to the drug suspension.

For suspension-based MDI formulation, it is preferred to have a narrow particle size distribution to avoid Ostwald ripening, which is essentially a process where the large particles grow at the expense of smaller particles. Ostwald ripening is a thermodynamically-driven process based on the principle that larger particles are more energetically favored than small particles. Surfactants or other surface-modifiers can also help to minimize or eliminate Ostwald ripening of the suspended drug particle in the pMDI.

The development of pMDI formulations has also be confronted with further challenges since the replacement of chlorofluorocarbons (CFCs) with the more environmentally friendly hydrofluoroalkane (HFA) propellants. In spite of the fact that the operation of pMDIs with HFAs is similar to those containing CFCs, previous formulations are generally not compatible due to differences in physiochemical properties between these two classes of fluids. One of the issues in reformulating or formulating pMDIs with HFAs is related to the fact that hydrocarbon-based surfactants used in FDA-approved CFC formulations (e.g., oleic acid, sorbitan trioleate, and lecithin) have extremely low solubility in the more polar semi-fluorinated propellants.

There is a significant need for stable orally inhaled benzodiazepines, such as lorazepam. The present invention satisfies this need.

### SUMMARY OF THE INVENTION

The invention encompasses methods and compositions of a pharmaceutical aerosol formulation comprising a particulate benzodiazepine and a propellant, wherein the aerosol formulation does not contain a surfactant or other surface modifiers. In one aspect, the pharmaceutical aerosol formulation is stable at 25° C. and 60% relative humidity (RH) conditions for at least 4 weeks after formulation. The particulate benzodiazepine is selected from the group consisting of alprazolam, bretazenil, bromazepam, brotizolam, chlordiazepoxide, cinolazepam, clonazepam, cloxazolam, clorazepate, delorazepam, diazepam, estazolam, flunitrazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, quazepam, temazepam, tetrazepam, triazolam and their pharmaceutically accepted salts. In another aspect, the particulate benzodiazepine is lorazepam or a pharmaceutically acceptable salt thereof. The propellant is selected from the group consisting of 1,1,1,2-tetrafluoroethane and 1,1,1,2,3, 3,3-heptafluoropropane. In another aspect, the propellant is 1,1,1,2,3,3,3-heptafluoropropane. The particulate benzodiazepine has a mean particle size of about 0.5 microns to about 3 microns. The particulate benzodiazepine has one or more of the following a d10 of about 0.5 micron to about 1.0 micron, a d50 of about 1.0 micron to about 2.0 microns, or a d90 of about 2 microns to about 3.0 microns. The pharmaceutical aerosol formulation is delivered to a subject using a pressured metered dose inhaler. The pressured meter dosed inhaler can be breath-actuated.

In another aspect, the invention encompasses a pharmaceutical aerosol formulation consisting of a particulate benzodiazepine and a propellant, wherein the aerosol formulation is stable at 25° C. and 60% relative humidity (RH) conditions for at least 4 weeks.

### BRIEF DESCRIPTION OF THE DRAWINGS

FIG. 1 depicts a 5 mg/ml micronized lorazepam in HFA227 formulation at time 0 (t=0) and at 1 minute after formulation (t=1 minute).

FIG. 2 is a graph depicting the dose uniformity of 5 mg/ml micronized lorazepam in HFA227 in aluminum canisters over time.

### DETAILED DESCRIPTION OF THE INVENTION

This detailed description of the invention is divided into sections for the convenience of the reader. Section I provides definitions of terms used herein. Section II provides a description of methods and compositions of excipient free, orally inhaled benzodiazepines. Section III provides a description of oral inhalation delivery systems. Section IV discloses examples that illustrate the various aspects and embodiments of the invention.

### I. DEFINITIONS

Unless defined otherwise, all technical and scientific terms used herein have the meaning commonly understood by a person skilled in the art to which this invention belongs. As used herein, the following terms have the meanings ascribed to them unless specified otherwise.

“Active pharmaceutical ingredient” or “API” refers to active chemical(s) used in the manufacturing of drugs. Another term synonymous with API is “bulk drug substance”.

“Colloid” refers to a chemical system composed of a continuous medium (continuous phase) throughout in which are distributed small particles (dispersed phase) that do not settle out under the influence of gravity. The particles may be in emulsion or in suspension.

“Creaming rate” refers to the time for flocs to form (i.e., to separate from the suspension) and rise to the top of the suspension. “Creaming” or when the flocs float to the top of the suspension usually occurs when the flocs have a lower density than the suspension. The counterpart to creaming rate is “sedimentation rate” which refers to the time for flocs to form and settle to the bottom of the formulation. The usually occurs when the flocs have a higher density than the suspension.

“Creaming volume” refers to the volume ratio of the flocculated particles relative to the whole formulation volume.

“Drug composition” or “drug formulation” refers to a composition comprising at least one API and at least one additional composition.

“Excipient” refers to pharmaceutically acceptable carriers that are relatively inert substances used to facilitate administration or delivery of an API into a subject or used to facilitate processing of an API into drug formulations that can be used pharmaceutically for delivery to the site of action in a subject. Non-limiting examples of excipients include stabilizing agents, surfactants, surface modifiers, solubility enhancers, buffers, encapsulating agents, antioxidants, preservatives, nonionic wetting or clarifying agents, viscosity increasing agents, and absorption-enhancing agents.

“Hydrofluorocarbon” refers to hydrofluoroalkanes (HFAs). In recent years, HFAs have replaced chloroflurocarbons (CFCs) as propellants due to environmental issues concerning the impact of CFCs on the earth's ozone layer. Examples of hydrofluoroalkane propellants include of 1,1,1,2-tetrafluoroethane (referred to as HFA134a) and 1,1,1,2,3,3,3-heptafluoropropane (referred to as HFA 227).

“Ostwald ripening” refers to the thermodynamically-driven process based on the principle that larger particles are more energetically favored than small particles.

“Particulate API” refers to an API that is manufactured at a desired particle size or particles of a desired particle size range.

“Stabilized pharmaceutical formulation” refers to a pharmaceutical formulation that exhibits physical and chemical stability in which the physical and chemical composition characteristics of the formulation do not change significantly due to the effects of time and temperature.

“Surface modifier” refers to organic or non-organic pharmaceutically acceptable excipients that are typically added to a drug formulation to alter formulation performance. Such alterations in performance include reduction, minimization or elimination of aggregation or agglomeration of particle of a drug. Surface modifiers include, but are not limited to, polymers, low molecular weight oligomers, and surfactants.

“Suspension” refers to a chemical system composed of components in a medium where the components are larger than those comprising the medium. Components of a suspension can be evenly distributed, for example by mechanical means, however, the components will settle out of the medium under the influence by gravity.

It is to be understood that this invention is not limited to particularly exemplified drug particles, formulations, or manufacturing processes parameters as such, may vary. It is also to be understood that the technical terminology used herein is for the purpose of describing particular embodiments of the invention only, and is not intended to be limiting.

### II. METHODS AND COMPOSITIONS OF EXCIPIENT-FREE, ORALLY INHALED BENZODIAZEPINES

### Benzodiazepines

One class of agents that are used to treat epileptic seizures is benzodiazepines. Benzodiazepines are a class of psychoactive drug that enhance the effect of the neurotransmitter gamma-aminobutyric acid (GABA). This class of drug has been used to treat anxiety, insomnia, agitation, seizure, muscle spasms, and as a sedative.

Epilepsy is a neurological disorder that is characterized by recurring seizures. These seizures are caused by abnormal increases in neuronal excitability due to the malfunction of membrane proteins that control the permeability of ions (i.e., sodium, potassium, calcium ions) across a neuron's membrane. Seizures are characterized by a change in sensation, awareness or behavior brought about by an electrical disturbance in the brain.

There are many different types of seizures that are experienced in epileptic patients. Physicians generally classify epileptic seizures on the basis of several factors, including the site of seizure origin, seizure frequency, and the electrophysiological property of the seizures, as well as in terms of response to therapy. The ILAE (International League Against Epilepsy) broadly characterizes seizures as either partial onset or generalized onset and this classification drives the majority of clinical treatment decisions.

Acute seizures are a serious medical crisis. Pharmacotherapy of epilepsy is not limited to chronic prophylactic management of seizures, but also the acute management of seizures, such as status epilepticus, acute repetitive seizures, seizures associated with post-anoxic insult, febrile seizures and alcohol withdrawal seizures (Riss et al., “Benzodiazepines in Epilepsy: Pharmacology and Pharmacokinetics” (2008) Acta Neurologica Scandinavica; v. 118(2), pp. 69-86). Status epilepticus is a prolonged state of persistent seizures—either one continual seizure over 30 minutes in duration or recurrent seizures without regaining consciousness in between—and is a major neurological emergency with an incidence of around 20 new cases per 100,000 people per year (Knake, et al., “Status Epilepticus: A Critical Review” (2009) Epilepsy & Behavior; v. 15, pp. 10-14). Mortality from status epilepticus can be as high as 40%. Acute repetitive seizures are distinct from status epilepticus: the condition refers to multiple seizures over a short period of time, such as 24 hours, but with periods of respite between the seizures. Prevalence has been estimated at 25 individuals per 100,000 people per year, occurring in around 3% of the epileptic population (Martinez, et al., “Prevalence of Acute Repetitive Seizures in the United Kingdom” (2009) Epilepsy Res.; v. 87(203), pp. 137-43). Benzodiazepines are among the most useful drugs available for treating status epilepticus, acute repetitive seizures and febrile seizures, given their high efficacy rates, fast onset and minimal toxicity.

Benzodiazepines such as lorazepam have been used to treat epilepsy and epileptic seizures. Lorazepam has generally been formulated either for oral administration or for intravenous (IV) or intramuscular (IM) administration. Because of its low water solubility, both injectable formulations (i.e., IV or IM) have been difficult to formulate. IM lorazepam has the further complication in that it has a slow onset of action compared to IV lorazepam. Oral formulations of lorazepam have disadvantages such as susceptibility to enzyme degradation in the gastrointestinal tract and oral delivery may have a slow absorption and onset of action. Oral inhalation delivery of benzodiazepines such lorazepam would overcome these difficulties and/or disadvantages.

Systemic delivery via the oral inhalation route (and thereby through absorption in the lungs into systemic circulation) provides several advantages when the primary intended site of action of the drug is the brain. One advantage is the very rapid absorption by the lung and delivery into systemic circulation. Once absorbed by the lungs, the drug will enter into the pulmonary artery and then to the carotid artery to the brain. Once in the brain, the drug can cross the blood-brain barrier and be delivered to the intended site of action. This targeted delivery to the brain avoids first pass metabolism and avoids any enzyme degradation that may occur. The targeted delivery also minimizes potential systemic side effects and may lower the dose required for efficacy in a subject. Because of the rapid onset of action achieved through pulmonary delivery of systemically active drugs, this method of delivery is preferred for acute treatment of symptoms. Also, unlike oral administration, pulmonary administration through oral inhalation bypasses the gastrointestinal tract and thus also avoids enzymatic degradation, problems with gastric stasis (in some diseases) and inconsistent absorption rates, giving the subject a more consistent delivery of the drug. Unlike IV or IM administration, pulmonary delivery is convenient, non-invasive, self-administrable and no hospitalization is required.

In some embodiments, the orally inhaled benzodiazepine is selected from the group consisting of alprazolam, bretazenil, bromazepam, brotizolam, chlordiazepoxide, cinolazepam, clonazepam, cloxazolam, clorazepate, delorazepam, diazepam, estazolam, flunitrazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, quazepam, temazepam, tetrazepam, triazolam and their pharmaceutically accepted salts. In some embodiments, the benzodiazepine is two or more combinations of the preceding list. In other embodiments, the benzodiazepine is lorazepam.

### Particle Generation

Particulate active pharmaceutical ingredient (API), such as particulate lorazepam, that are of an acceptable particle size for delivery to the lungs in an aerosol formulation may be generated in a variety of manner. For illustrative purposes, API particles may be generated from the bulk API by attrition processes such as grinding, micronizing, milling or the like. API particles may also be generated through a multiphase precipitation process such as spray drying, solution precipitation, in situ precipitation, volume exclusion precipitation, supercritical extraction/precipitation, lyophilization, or the like. API particles for use in aerosols are generally manufactured to a size of about 0.05 microns to about 10 microns, of about 0.1 microns to about 5 microns, of about 0.5 microns to about 3 microns, and of about 1 micron to about 3 microns. In various embodiments, the active pharmaceutical ingredient has a particle size in the range of about 0.5 microns to about 3 microns. In other embodiments, the API has a particle size in range of about 1 micron to about 3 microns.

### Formulation for Oral Inhalation Delivery

Inhalation aerosols of drug formulation for delivery using a pressurized metered dose inhaler typically include excipients such as surfactants and other surface modifiers to increase the stability of the particles or to increase the deliverability of these drugs in an aerosol form. However, excipients such as surfactants and other surface modifiers have been associated with toxicity in the subject and other undesirable side effects. To avoid such toxicity problems, the drug formulation of the present invention is free of excipients such as surfactants and other surface modifiers.

The drug formulation may include one or more active pharmaceutical ingredient in any appropriate amount (singularly or in aggregate). In some embodiments, the API(s) may be selected to be in a certain concentration in order to achieve a desired concentration(s) after delivery into the subject or patient. In other embodiments, the API(s) may be selected to be in a certain concentration to conform to a certain dosing regimen or to achieve a certain desired effect.

In some embodiments, the active pharmaceutical ingredient in the formulation is a benzodiazepine. In some embodiments, the active pharmaceutical ingredient in the formulation is lorazepam. In other embodiments, the formulation comprises a concentration of lorazepam wherein a delivered dose of the formulation achieves systemic plasma concentration of Cmax levels of about 30 ng/ml to 40 ng/ml in the subject.

In inhaled aerosol drug formulations, colloidal stability is a desired characteristic. In some cases, aerosol delivery of the API comprises the use of a propellant in the formulation. In such cases, the propellants may take a variety of forms. In a non-limiting example, the propellant may be a compressed gas or a liquefied gas. Chlorofluorocarbons (CFCs) were once commonly used as liquid propellants, but have now been banned due to the negative impact on the earth's ozone layer. They have been replaced by the now widely accepted hydrofluorocarbon or hydrofluoroalkane (HFA) propellants. The most commonly used HFAs are 1,1,1,2-tetrafluoroethane, which is also referred to as 134a or HFA134a; and 1,1,1,2,3,3,3-heptafluoropropane, which is also referred to as 227 or HFA227, both available from Dupont, Solvay Chemicals or Mexichem Fluor. In some cases, the propellant can be one HFA compound or a mixture of two or more HFA compounds.

In some embodiments, the propellant is selected from the group consisting of 1,1,1,2-tetrafluoroethane and 1,1,1,2,3,3,3-heptafluoropropane. In other embodiments, the propellant is 1,1,1,2-tetrafluoroethane. In some embodiments, the propellant is 1,1,1,2,3,3,3-tetrafluoropropane. In some embodiments, the propellant is a mixture of 1,1,1,2-tetrafluoroethane and 1,1,1,2,3,3,3-tetrafluoropropane.

While HFA propellants are more environmentally friendly, these propellants have significant solvency challenges. In general, the HFA propellants have lower solvency characteristics as compared to their CFC predecessors. Because of the solvency issue, more and more pMDI formulations are turning to a suspension-based formulation instead of a solution-based formulation. In a suspension-based formulation, the drug particles are usually maintained as a colloid suspension. Even as a suspension-based formulation, it is a significant challenge to obtain a stable colloidal suspension of the API in the select propellant/propellant mix.

For suspension-based MDI formulation, it is preferred to have a narrow particle size distribution to avoid Ostwald ripening, which is essentially a process where the large particles grow at the expense of smaller particles. Ostwald ripening is a thermodynamically-driven process based on the principle that larger particles are more energetically favored than small particles. Ostwald ripening can increase the particle size over time and thus deteriorate the aerosol performance of the formulation. The Ostwald ripening effect can be minimized by using drug particles such as benzodiazepine particles with a narrow size distribution. In suspension-based aerosol formulation, it is preferred to have a size distribution of d₁₀ of about 0.5 micron to about 1.0 micron, d₅₀ of about 1 micron to about 2 microns, and d₉₀ of about 2 microns to about 3 microns.

Stability of a suspension-based MDI formulation can be determined by a variety of methods. One such method is to measure the fine particle dose (FPD) over time in different temperature/humidity conditions. Initially, the formulation will see a drop in FPD, but the FPD should remain substantially unchanged after this initial drop if the aerosol formulation is stable. In contrast, in an unstable aerosol formulation, and therefore undesirable, the FPD will continue to decrease over time. A stable aerosol formulation will have a FPD that remain substantially unchanged after the initial drop at conditions of 25° C. and 60% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation, or at least 6 months after formulation. In other embodiments a, stable aerosol formulation will have a FPD that remain substantially unchanged after the initial drop at accelerated conditions of 40° C. and 75% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation, or at least 6 months after formulation.

Another measure of stability is the measure of the mass median aerodynamic diameter (MMAD) of the particles over time. Initially, the formulation will see an increase in MMAD, but then the MMAD should plateau (coinciding with a stabilization of FPD) after this initial increase. A stable aerosol formulation will have a MMAD that remain substantially unchanged after the initial increase at conditions of 25° C. and 60% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation, or at least 6 months after formulation. In other embodiments a, stable aerosol formulation will have a MMAD that remain substantially unchanged after the initial increase at accelerated conditions of 40° C. and 75% relative humidity (RH) for at least 3 weeks after formulation, at least 4 weeks after formulation, at least 5 weeks after formulation, at least 6 weeks after formulation, at least 8 weeks after formulation, at least 10 weeks after formulation, at least 12 weeks after formulation, or at least 6 months after formulation.

### III. ORAL INHALATION DELIVERY SYSTEMS

A wide variety of delivery methods/platforms are suitable for the practice of the invention. Inhalation devices or other non-injectable devices are preferred devices and function by delivering an aerosol of the drug formulation into the subject or patient. These inhalation devices generally including a housing having a proximal end and a body portion. A mouthpiece or nose piece will typically be positioned at the proximal end. In another variation, the inhalation device may be a pressurized metered dose inhaler (pMDI) with the drug composition adjusted to generate a significant portion of the delivered dose in the respirable range (free drug or drug contained in propellant droplets having sizes less than approximately 5 micron, preferably from about 2 microns to about 3 microns). In some variations, the pMDI can be fitted with nosepiece adapters or facemask adaptors to administer the drug laden propellant to the nasopharynx for better and/or more efficient deliver.

### Pressurized Metered Dose Inhalers

Pressurized metered dose inhalers (pMDIs) generally have two components: a canister in which the drug composition particles are stored under pressure in a suspension or solution form; and a receptacle used to hold and actuate the canister. The canister may contain multiple doses of the drug composition, although it is possible to have single dose canisters as well. The canister may include a valve, from which the contents of the canister may be discharged. In some embodiments, the valve is a metering valve. Aerosolized drug composition is dispensed from the pMDI by applying a force on the canister to push it into the receptacle, thereby opening the valve and causing the drug particles to be conveyed from the valve through the receptacle outlet. Upon discharge from the canister, the drug composition particles are atomized, forming an aerosol. pMDIs generally use propellants to pressurize the content of the canister and to propel the drug particles out of the receptacle outlet. In pMDIs, the drug composition is provided in liquid form, and resides within the canister along with the propellant.

In some instances, a manual discharge of aerosolized drug must be coordinated with inhalation, so that the drug composition particles are entrained within the inspiratory air flow and conveyed to the lungs. In other instances, a breath-actuated trigger, such as that included in the Tempo Inhaler® (MAP Pharmaceuticals, Mountain View, Calif.) may be employed that simultaneously discharges a dose of drug upon sensing inhalation. Such breath-actuated pMDI automatically discharges the drug composition aerosol at the appropriate time during inhalation by the user or subject. These devices are generally known as breath-actuated pressurized metered dose inhalers (baMDIs).

All references cited herein are incorporated by reference in their entireties, whether previously specifically incorporated or not. The publications mentioned herein are cited for the purpose of describing and disclosing reagents, methodologies and concepts that may be used in connection with the present invention. Nothing herein is to be construed as an admission that these references are prior art in relation to the inventions described herein.

Although this invention has been described in connection with specific embodiments thereof, it will be understood that it is capable of further modifications. This application is intended to cover any variations, uses, or adaptations of the invention following, in general, the principles of the invention and including such departures from the present disclosure as come within known or customary practice within the art to which the invention pertains and as may be applied to the essential features hereinbefore set forth.

The following examples serve to more fully describe and exemplify the above disclosed embodiments. It is understood that these examples in no way serve to limit the true scope of this disclosure, but rather are presented for illustrative purposes.

### IV. EXAMPLES

### Example 1

### Visual Appearance of Excipient-Free Lorazepam Suspension in HFA

Micronized lorazepam and pharmaceutical-grade hydrofluoroalkane 227 (HFA 227) were purchased from Cambrex (Italy) and Mexichem (Mexico), respectively.

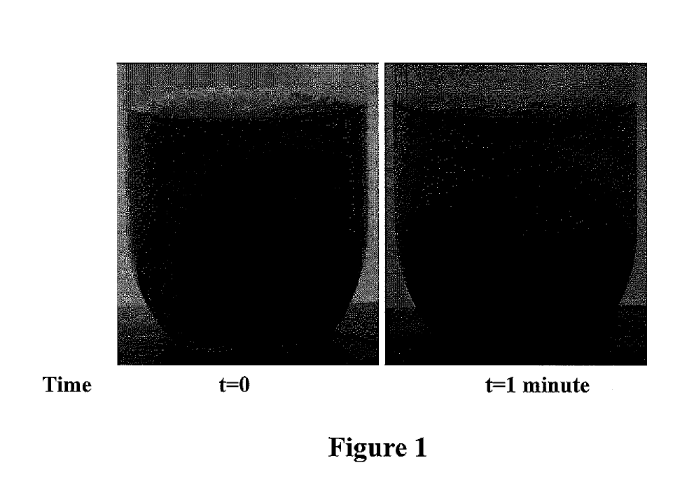

Micronized lorazepam was formulated in HFA227 at a concentration of 5 mg/ml. At t=0, flocs begin to form. At t=1 minute, loose agglomerates are present. As shown in FIG. 1, the excipient-free formulation was very “fluffy” with loose agglomerates that were redispersed easily by manual shaking.

### Example 2

### Dose Uniformity of Excipient-Free Lorazepam Suspension in HFA

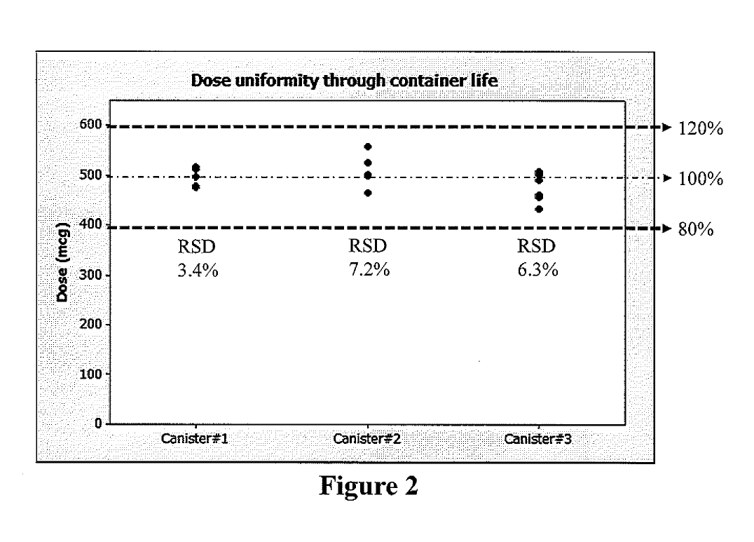

To test the dose uniformity of excipient-free lorazepam, micronized lorazepam was formulated in HFA227 at a concentration of 5 mg/ml without any other excipients. Three separate 5.9 ml plain aluminum canisters were filled with 5 mg/ml lorazepam/HFA formulation and tested for dose uniformity through container life. The results, shown in FIG. 2, showed excellent dose uniformity through container life and met FDA requirements.

### Example 3

### Excipient-Free Lorazepam Suspension Stability

The stability of the excipient-free lorazepam suspension formulation was tested over a period of 6 weeks at 25° C./60% relative humidity (RH) and at 40° C./75% RH. Micronized lorazepam was formulated in HFA227 at a concentration of 5 mg/ml. The stability results showed that the fine particle dose (FPD) of the formulation is decreased and mass median aerodynamic diameter (MMAD) is increased over time at both storage conditions. The decrease in formulation performance over time is mainly attributed to Ostwald ripening. The broad particle size distribution of the lorazepam particles in the formulation (with d₁₀=0.34 micron; d₅₀=1.85 micron; and d₉₀=5.82 micron) is most likely the result of Ostwald ripening.

We claim:

1. A pharmaceutical aerosol formulation comprising: (i) a particulate benzodiazepine; and (ii) a propellant, wherein the aerosol formulation does not contain a surfactant or other surface modifiers.

2. The pharmaceutical aerosol formulation of claim 1, wherein the formulation is stable at 25° C. and 60% relative humidity (RH) conditions for at least 4 weeks.

3. The pharmaceutical aerosol formulation of claim 1, wherein the benzodiazepine is selected from the group consisting of alprazolam, bretazenil, bromazepam, brotizolam, chlordiazepoxide, cinolazepam, clonazepam, cloxazolam, clorazepate, delorazepam, diazepam, estazolam, flunitrazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, quazepam, temazepam, tetrazepam, triazolam and their pharmaceutically accepted salts.

4. The pharmaceutical aerosol formulation of claim 3, wherein the benzodiazepine is lorazepam or a pharmaceutically acceptable salt thereof.

5. The pharmaceutical aerosol formulation of claim 1, wherein the propellant is selected from the group consisting of 1,1,1,2-tetrafluoroethane and 1,1,1,2,3,3,3-heptafluoropropane.

6. The pharmaceutical aerosol formulation of claim 5, wherein the propellant is 1,1,1,2,3,3,3-heptafluoropropane.

7. The pharmaceutical aerosol formulation of claim 1, wherein the benzodiazepine has a mean particle size of about 0.5 microns to about 3 microns.

8. The pharmaceutical aerosol formulation of claim 1, wherein the particulate benzodiazepine has one or more of the following:
(i) a d₁₀ of about 0.5 micron to about 1.0 micron;
(ii) a d₅₀ of about 1.0 micron to about 2.0 microns; or
(iii) a d₉₀ of about 2.0 microns to about 3.0 microns.

9. The pharmaceutical aerosol formulation of claim 1, wherein the formulation is delivered to a subject using a pressured metered dose inhaler.

10. The pharmaceutical aerosol formulation of claim 9, wherein the pressured metered dose inhaler is breath-actuated.

11. A pharmaceutical aerosol formulation consisting of a particulate benzodiazepine and a propellant, wherein the aerosol formulation is stable at 25° C. and 60% relative humidity (RH) conditions for at least 4 weeks.
